(19) United States
(12) Patent Application Publication
(10) Pub. No.: US 2016/0007908 A1
(43) Pub. Date: Jan. 14, 2016

(54) HAIR TESTING DEVICE AND HAIR TESTING SYSTEM

(71) Applicant: BOE TECHNOLOGY GROUP CO., LTD., Beijing (CN)

(72) Inventor: Weiqing GUO, Beijing (CN)

(21) Appl. No.: 14/436,927

(22) PCT Filed: Sep. 26, 2014
(86) PCT No.: PCT/CN2014/087520

§ 371 Date: Apr. 20, 2015

(30) Foreign Application Priority Data
Feb. 20, 2014 (CN) 201410058104.8

(51) Int. Cl. A61B 5/00; G01N 21/17; G01N 33/483

(52) U.S. Cl. CPC A61B 5/448; G01N 33/4833; G01N 21/17; A61B 5/004; A61B 5/0077; A61B 5/6814; A61B 5/742; G01N 2021/8444

(57) ABSTRACT

The present invention provides a hair testing device and a hair testing system. The hair testing device includes a data acquisition part and a transmitting part. The data acquisition part is configured to acquire hair-related data and send the acquired data to the transmitting part. The transmitting part is configured to receive the acquired data and transmit the acquired data to a data processing device which is capable of processing data. The hair testing device further includes a fixing part for fixing to hair, and the data acquisition part and the transmitting part are both located on the fixing part. The hair testing device provided by the present invention is capable of automatically acquiring hair-related data, and sending the acquired data to the data processing device for processing, so as to achieve automatic testing of hair quality.

### FIELD OF THE INVENTION

The present invention relates to the field of hair quality testing technology, and particularly relates to a hair testing device and a hair testing system.

### BACKGROUND OF THE INVENTION

Hair is outward manifestation of human health. Hair quality of a person can directly reflect his health condition. For example, dark black, thick, glossy, lustrous hair may represent good physical health condition of a person, while coarse, split, dry, yellow, thin hair is not only indicative of the need of hair care, but also likely to reflect internal physical problems, and must be regarded seriously. Thus, people have paid more and more attention to the change of their hair quality.

A hair testing device is an instrument for testing hair quality, with which people can better know their hair condition by the testing of indicators in various aspects of hair so as to learn their physical health condition. Hair testing devices in the prior art are generally large machines, and mainly applied in hairdressing and hair care industry. Consumers can test their hair only when they go to such a specific place. This cannot meet people's demand of daily testing of their hair quality. In spite to the advent of some small-size hair testing devices that can be used every day at home, such household testing devices usually need complicated manual operation and consume a relatively long time, and cannot achieve an automatic monitoring of hair quality.

Accordingly, there is an urgent need of a hair testing device capable of automatically monitoring hair quality.

### SUMMARY OF THE INVENTION

An object of the present invention is providing a hair testing device capable of automatically monitoring hair quality.

A technical solution for achieving the object of the present invention is as follows: a hair testing device includes a data acquisition part and a transmitting part, wherein the data acquisition part is configured to acquire hair-related data and send the acquired data to the transmitting part, and the transmitting part is configured to receive the acquired data from the data acquisition part and transmit the acquired data to a data processing device which is capable of processing data; the hair testing device further includes a fixing part for fixing to the hair, and the data acquisition part and the transmitting part are located on the fixing part.

Preferably, in the hair testing device, the data acquisition part is a sensor, and the transmitting part is a signal emitter.

Preferably, in the hair testing device, the sensor is an image sensor.

Preferably, in the hair testing device, the image sensor is a CCD image sensor or CMOS image sensor.

Preferably, in the hair testing device, the fixing part is a hair sticker.

Preferably, in the hair testing device, the hair sticker is provided with a flexible printed circuit board, on which the transmitting part is located.

Preferably, an LED lamp is provided in the hair testing device at a side close to the hair, and is used for illuminating the hair when the image sensor performs image acquisition.

The present invention has the following beneficial effects:

When the hair testing device provided by the present invention is worn on hair and started, it can acquire hair-related data automatically, and send the acquired data to the data processing device which is capable of processing data, so as to achieve automatic testing of hair quality. Furthermore, as the hair testing device provided by the present invention is provided with the fixing part, it may be worn directly on the hair without affecting user's other activities, thus providing a more convenient way of testing.

To achieve the object of the present invention, a hair testing system is further disclosed, including a hair testing device, a data processing device and a display device, wherein the hair testing device is the above-mentioned hair testing device;

the data processing device is configured to receive data acquired by the hair testing device, process the acquired data to obtain a processing result, and send the processing result to the display device; and

the display device is configured to receive and display the processing result.

Preferably, in the hair testing device, the data processing device includes a storage unit and a comparison and analysis unit; the storage unit is configured to store reference values of hair parameters; the comparison and analysis unit is configured to obtain current values of the hair parameters according to the acquired data received by the data processing device, compare the current values with the reference values to obtain difference values, and determine hair quality from the difference values.

Preferably, in the hair testing device, the comparison and analysis unit grades the difference values and determines the hair quality according to the graded difference values.

Preferably, in the hair testing device, the data processing device is provided on the fixing part; or the data processing device is provided on an external computer or mobile phone.

Preferably, in the hair testing device, the display device is provided on the fixing part; or the display device is provided on an external computer or mobile phone.

Preferably, in the hair testing device, the display device is a display screen.

The hair testing system provided by the present invention includes the hair testing device, which achieves the testing function by cooperating with the data processing device and the display device.

### BRIEF DESCRIPTION OF THE DRAWINGS

FIG. 1 is a schematic functional block diagram of a hair testing system provided by an embodiment of the present invention;

FIG. 2 is a structural schematic diagram of a fixing part of a hair testing device provided by an embodiment of the present invention; and

FIG. 3 is a schematic functional block diagram of a data processing device provided by an embodiment of the present invention.

### DETAILED DESCRIPTION OF THE EMBODIMENTS

To make a person skilled in the art better understand the technical solution of the present invention, the present invention is further described below in details in conjunction with the accompanying drawings and the specific implementations.

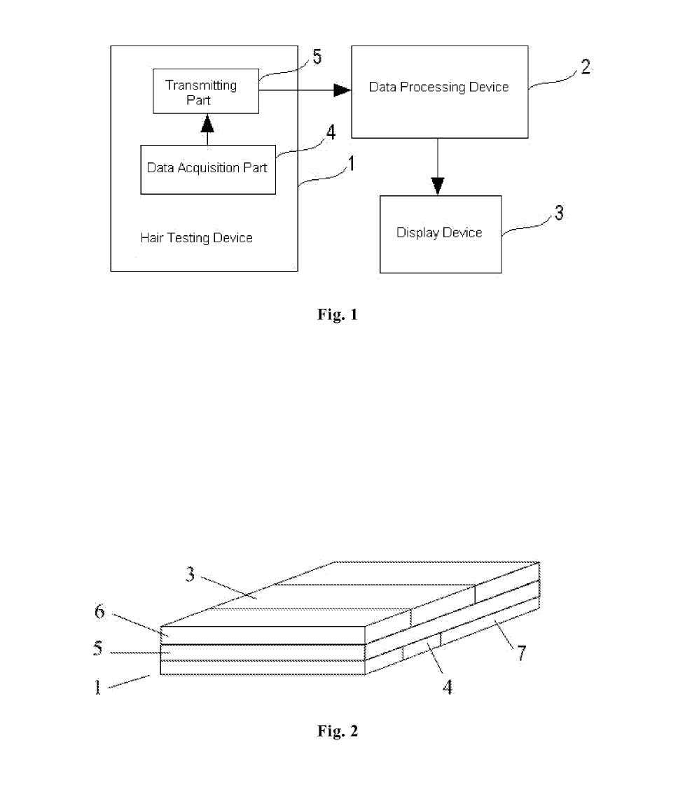

Referring to FIGS. 1 and 2, FIG. 1 is a schematic functional block diagram of a hair testing system provided by an embodiment of the present invention; and FIG. 2 is a structural schematic diagram of a fixing part of a hair testing device provided by an embodiment of the present invention.

As shown in FIG. 1, a hair testing device 1 provided by the present invention includes a data acquisition part 4 and a transmitting part 5, wherein the data acquisition part 4 is configured to acquire hair-related data and send the acquired data to the transmitting part 5, and the transmitting part 5 is configured to receive the data acquired by the data acquisition part 4 and transmit the acquired data to a data processing device 2 which is capable of processing data, in order to process the acquired data by the data processing device 2. The hair testing device 1 further includes a fixing part 7 for fixing itself to hair (see FIG. 2), and the data acquisition part 4 and the transmitting part 5 may be located on the fixing part 7.

When the hair testing device 1 provided by the present invention is worn on hair and started up, the hair testing device 1 starts acquiring hair-related data automatically, and sends the acquired data to the data processing device 2 with data processing function, so as to achieve automatic testing of hair quality. Furthermore, as the hair testing device 1 provided by the present invention is provided with the fixing part 7, it may be worn directly on hair without affecting user's other activities, thus providing a more convenient way of testing.

Specifically, the data acquisition part 4 may be a sensor, and the transmitting part 5 may be a signal emitter.

In a preferred embodiment, the data acquisition part 4 may be (but not limited to) an image sensor, through which an image of hair is taken, and the image is sent, e.g. in wireless method, to the data processing device 2 for processing. Specifically, the image sensor may be a CCD image sensor or CMOS image sensor. The CCD (Charge-coupled Device) image sensor may convert an optical signal to an analog current signal directly, and perform amplification and analog-digital conversion on the obtained current signal, so as to achieve acquirement, storage, transmission, processing and reproduction of the image. A chip of the CMOS (Complementary Metal Oxide Semiconductor) image sensor involves a CMOS process, and thus is featured by high integration. When the CMOS image sensor is used, an image acquisition circuit and a signal processing circuit may be integrated to the same chip, so that the hair testing device including the CMOS image sensor may be smaller in size.

Selection of parameters of the image sensor will be described below. Important parameters of the image sensor include resolution and lens focal length.

The calculation formula of the resolution of the image sensor is as follows:

S=(A/D)×2 (1)

In formula (1), S is the resolution of the image sensor, A is the size of a testable area (data acquirable range), and D is a target minimal feature (size of a testable minimal feature).

Supposing S=30 k, and A=5 mm, then according to formula (1), we can obtain:

D=(A/S)×2=(5/30)×2=0.33 μm.

As the diameter of hair is about 40-100 μm, in case of the above-mentioned parameters, the target minimal feature D is 0.33 μm, which is far smaller than 40 μm, thus the requirement of testing hair thickness and split can be fully met even with the resolution S=30 k.

The calculation formula of the lens focal length is as follows:

L1×A=B×L2 (2)

In formula (2), L1 is the lens focal length, A is the size of the testable area, B is the size of the image sensor, and L2 is an object distance. Formula (2) can be transformed to:

L1=(B×L2)/A (3)

Supposing B=10 mm, L2=1 mm, and A=5 mm, then according to formula (3), we can obtain:

L1=(10×1)/5=2 mm.

That is, a lens with a focal length L1=2 mm can meet the requirement of the hair testing device of the present invention.

To sum up, an image sensor with a resolution of 30 k and a focal length of 2 mm may be adopted as the data acquisition part in the hair testing device of the present invention.

In the embodiment of the present invention, the fixing part 7 may be a small and light-weight part that is convenient to fix to hair, such as a hair sticker. As shown in FIG. 2, the fixing part 7 which is a hair sticker is shaped as a flat cuboid, with densely distributed barbs on one surface, for directly attaching to hair in order to play the role of fixing the hair testing device 1 relative to hair. An advantage of the hair sticker lies in that hair would not be deformed even if such hair testing device 1 is fixedly worn through the hair sticker for a long time. In this embodiment, the data acquisition part 4 (which may be specifically a sensor) is arranged in the hair sticker and can contact with hair. That is, the data acquisition part 4 is located on a surface of the hair sticker on which the barbs is provided. Thus, when the hair sticker is attached to hair, the data acquisition part 4 can directly contact with hair to facilitate data acquisition. Furthermore, the transmitting part 5 (which may be specifically a signal emitter) may be arranged on the other surface of the hair sticker being opposite to the surface provided with barbs. After acquiring data, the data acquisition part 4 may directly send the acquired data to the transmitting part 5. As the fixing part 7 is a small and light-weight part, such as a hair sticker, that is convenient to fix to hair, and both the data acquisition part 4 and the transmitting part 5 may be composed of highly integrated electronic devices, the hair testing device 1 of the present invention is very light, small and exquisite as a whole, and comfort in wearing is improved.

In a preferred embodiment, the main body of the hair sticker is made of a flexible material, and the hair sticker is provided with a flexible printed circuit board (FPCB), on which are located the data acquisition part 4 and the transmitting part 5. The flexible printed circuit board is a patterned printed circuit board made of a flexible substrate, and thus has the characteristics of pliability and elastic deformation. After being attached to hair, the hair sticker is deformed somewhat due to curvature of the head. The flexible printed circuit board can also be deformed with the hair sticker due to the above-mentioned characteristics, thus improving comfort feeling of the user wearing the hair testing device 1 of the present invention.

Of course, the fixing part 7 may also take other form of implementation, such as a hair band, a hair pin or the like, as long as a carrier is provided for the data acquisition part 4 and the transmitting part 5 while playing the role of fixing the hair testing device 1 to hair.

Further, in consideration of aesthetics, in this embodiment, an outer layer ornament 6 is also provided on the FPCB.

In a preferred embodiment, an LED lamp is arranged in the hair testing device 1 at a side close to hair, and is used for illuminating a photographed hair area when the data acquisition part 4 (which may be specifically an image sensor) picks up an image.

The present invention also discloses a hair testing system, including a hair testing device, a data processing device and a display device, wherein the hair testing device is the above-mentioned hair testing device 1. The data processing device is configured to receive the acquired data sent by the hair testing device, process the acquired data to obtain a processing result, and send the processing result to the display device. The display device is configured to receive the processing result sent by the data processing device, and display the processing result.

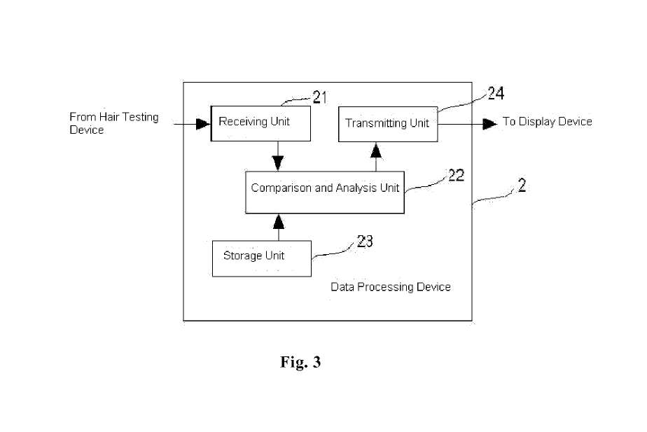

The data processing device 2 of the present invention is described below with reference to FIG. 3.

FIG. 3 is a schematic functional block diagram of the data processing device provided by an embodiment of the present invention. The data processing device 2 may be any device with a data processing function, such as a microprocessor, a microcomputer or the like. As shown in FIG. 3, the data processing device 2 includes a receiving unit 21, a comparison and analysis unit 22, a storage unit 23 and a transmitting unit 24. The receiving unit 21 is configured to receive hair-related data acquired by the hair testing device 1, and transmit the received data to the comparison and analysis unit 22. The receiving unit 21 may be, for example, a wireless receiver. For example, the hair-related data may be images of some hair or the like acquired by an image sensor, and may also be data such as hair density, hair oily degree or the like acquired by other type of data acquisition device. The present invention is not limited thereto. The comparison and analysis unit 22 may obtain values of various parameters reflecting hair condition (such as hair color, scurf density and the like) according to the data-related data, and compare the obtained values of parameters about hair (current values of the hair parameters) with reference values of corresponding parameters about hair pre-stored in the storage unit 23, in order to determine health condition of hair. The storage unit 23 may store the reference values of the parameters mentioned above, thresholds or upper and lower limit values of ranges for determination, and the like. The transmitting unit 24 may transmit a determining result obtained by the comparison and analysis unit 22 to the display device 3 for display. The transmitting unit 24 may additionally transmit tested parameter values and reference values of corresponding parameters while transmitting the determining result, in order to assist the user to know about the hair conditions in details. The transmitting unit 24 may be, for example, a wireless transmitter. The transmitting unit 24 may be integrated with the receiving unit 21 into a transceiver.

Specifically, the comparison and analysis unit 22 compares the current parameter values with the reference values to obtain difference values, and determines hair quality from the difference values.

For example, as to testing of hair color, the data processing device 2 compares a color value in the acquired data and a reference value of color to obtain a difference value therebetween. When the difference value is located within a certain range, the color of hair is determined as good, otherwise, the color of hair is determined as bad. Further, in order to refine the processing result, the difference value may also be graded to obtain different color levels. Condition of other parameters (such as hair luster) of hair may be determined in a similar manner. Subsequently, the comparison and analysis unit 22 may perform comprehensive determination on the health condition of hair according to comparison results of multiple parameters, or may also simply determine the health condition of hair only according to a comparison result of one parameter, so as to obtain a final health condition result for reference available to the user.

It may be understood that reference values of the parameters stored in the storage unit 23 may be critical values or standard values representing health condition obtained in a medical or statistical way. For example, a reference value may depend on a health-related value available in the medical field. Furthermore, a sample mean value of many people obtained by a sampling survey may be used as a reference value. For example, a plurality of images may be taken at the same position of hair by an image sensor, and the images are sent to the data processing device 2 to extract image data of the same area, and analysis and comparison are conducted to obtain an average value, which is a sample value of hair average condition.

The hair testing system provided by the embodiment of the present invention can achieve automatic testing of hair quality merely by fixing the fixing part of the hair testing device to hair without causing any adverse influence on user's other activities. Thus, the hair testing system is very convenient to operate and carry, and may be popularized in a large scale for collecting sample values, to obtain reference values of a great many people in a certain region, so that the reference values are more worthy of reference.

In the embodiment of the present invention, position of the display device 3, which is configured to receive and display the processing result of the data processing device 2, is not limited. The display device 3 may be directly provided on an external computer or mobile phone, and may also be provided in the hair testing device 1 of the present invention, such as on the fixing part 7. Preferably, the display device 3 is provided on the hair sticker. In a specific example, the display device 3 is provided above the FPCB of the hair sticker, and receives by a wireless receiver the processing result transmitted by the transmitting unit 24 of the data processing device 2.

Specifically, the display device 3 is a display screen, for visually displaying the processing result. Of course an LED lamp may also be adopted, and the condition of tested hair quality is indicated by flickering of lamp light or different colors of the lamp.

The data processing device 2 is not limited in location, and may be provided on an external computer to facilitate operating and storing testing data by the user, and may also be provided on a mobile phone, IPAD or other portable external device to facilitate carrying around; in addition, it may also be arranged on the fixing part 7.

To sum up, the hair testing device provided by the present invention may test hair quality in real time while being fixed to hair, in order to achieve testing of the parameters of hair roughness, color, luster, cleanliness and the like. Furthermore, the hair testing device of the present invention is small in size, and easy to carry. The hair testing system provided by the present invention includes the hair testing device, which achieves the testing function by cooperating with the data processing device and the display device.

It should be understood that the above implementations are only exemplary implementations for illustrating the principle of the present invention, however, the present invention is not limited thereto. Many variations and improvements can be made by the person of ordinary skill in the art without departing from the spirit and essence of the present invention, and these variations and improvements should also be considered to be within the protection scope of the present invention.

1-13. (canceled)

14. A hair testing device, comprising a data acquisition part and a transmitting part, wherein the data acquisition part is configured to acquire hair-related data and send the acquired data to the transmitting part, and the transmitting part is configured to receive the acquired data from the data acquisition part and transmit the acquired data to a data processing device which is capable of processing data, the hair testing device further comprising a fixing part for fixing to the hair, and the data acquisition part and the transmitting part being located on the fixing part.

15. The hair testing device according to claim 14, wherein the data acquisition part is a sensor, and the transmitting part is a signal emitter.

16. The hair testing device according to claim 15, wherein the sensor is an image sensor.

17. The hair testing device according to claim 16, wherein the image sensor is a CCD image sensor or CMOS image sensor.

18. The hair testing device according to claim 16, further comprising an LED lamp being arranged on a side close to the hair, and used for illuminating the hair when the image sensor performs image acquisition.

19. The hair testing device according to claim 14, wherein the fixing part is a hair sticker.

20. The hair testing device according to claim 15, wherein the fixing part is a hair sticker.

21. The hair testing device according to claim 16, wherein the fixing part is a hair sticker.

22. The hair testing device according to claim 17, wherein the fixing part is a hair sticker.

23. The hair testing device according to claim 18, wherein the fixing part is a hair sticker.

24. The hair testing device according to claim 19, wherein the hair sticker is provided with a flexible printed circuit board, on which the transmitting part is located.

25. A hair testing system, comprising a hair testing device, a data processing device and a display device, wherein:
the hair testing device is the hair testing device of claim 14;
the data processing device is configured to receive data acquired by the hair testing device, process the acquired data to obtain a processing result, and send the processing result to the display device; and
the display device is configured to receive and display the processing result.

26. The hair testing system according to claim 25, wherein the data acquisition part is a sensor, and the transmitting part is a signal emitter.

27. The hair testing system according to claim 26, wherein the sensor is an image sensor.

28. The hair testing system according to claim 27, wherein the hair testing device further comprising an LED lamp being arranged on a side close to the hair, and used for illuminating the hair when the image sensor performs image acquisition.

29. The hair testing system according to claim 25, wherein the data processing device includes a storage unit and a comparison and analysis unit; the storage unit is configured to store reference values of hair parameters; the comparison and analysis unit is configured to obtain current values of the hair parameters according to the acquired data received by the data processing device, compare the current values with the reference values to obtain difference values, and determine hair quality from the difference values.

30. The hair testing system according to claim 29, wherein the comparison and analysis unit grades the difference values and determines the hair quality according to the graded difference values.

31. The hair testing system according to claim 25, wherein the data processing device is provided on the fixing part;
or the data processing device is provided on an external computer or mobile phone.

32. The hair testing system according to claim 25, wherein the display device is provided on the fixing part;
or the display device is provided on an external computer or mobile phone.

33. The hair testing system according to claim 32, wherein the display device is a display screen.
